(19) United States
(12) Patent Application Publication
(10) Pub. No.: US 2013/0225944 A1
(43) Pub. Date: Aug. 29, 2013

(54) FLEXIBLE SENSOR ASSEMBLY

(71) Applicant: STMicroelectronics S.r.l., (US)

(72) Inventors: Maurizio GRECO, Acicastello (IT); Antonio Maria SCALIA, Catania (IT)

(73) Assignee: STMicroelectronics S.r.l., Agrate (IT)

(21) Appl. No.: 13/776,069

(22) Filed: Feb. 25, 2013

(30) Foreign Application Priority Data
Feb. 28, 2012 (IT) TO2012A000177

(51) Int. Cl. A61B 5/145; A61B 5/01; A61B 5/1468; A61B 5/04

(52) U.S. Cl. CPC A61B 5/14532; A61B 5/04; A61B 5/01; A61B 5/14539; A61B 5/1468
USPC 600/301; 600/300; 600/365; 29/595

(57) ABSTRACT

A sensor assembly includes: an electrically insulating and flexible body (13), in the form of a plate having a first face (13a) and a second face (13b); and a first sensor (15; 315) and a second sensor (17), which are incorporated in the body (13). The first sensor (15) is set between the first face (13a) and the second sensor (17), and the second sensor (17) is set between the first sensor (15) and the second face (13b).

### PRIOR RELATED APPLICATIONS

This invention claims priority to Italian application TO2012A000177, filed on Feb. 28, 2012, and incorporated by reference in its entirety herein.

### FEDERALLY SPONSORED RESEARCH STATEMENT

Not applicable.

### FIELD OF THE INVENTION

The present invention relates to a flexible sensor assembly and to a process for manufacturing a flexible sensor assembly.

### BACKGROUND OF THE INVENTION

The use of biosensors, for example, for carrying out measurements in vivo of clinical parameters, is becoming increasingly widespread. This is in part due to the continuous development of technology in the area of microelectronics, which enables, amongst other things, extreme levels of miniaturization to be achieved.

The treatment of many pathological conditions draws great benefit from the increasing availability of biosensors for measurements in vivo with contained cost and a high degree of reliability. Diabetes, for example, represents one of the most important pathological conditions on a global level. It is the cause of numerous deaths, and there has been recorded a progressive increase in its incidence in the population. Notwithstanding the potential seriousness, diabetes can be effectively countered with timely administration of drugs, preventing onset of complications, if there is available the possibility of carrying out frequent, fast, and accurate measurements of the blood-glucose level. Glucometers are portable devices of contained cost that meet this need.

A glucometer normally comprises a sensor, housed in a polymeric cannula or in a needle, and a processing unit coupled to the sensor. Normally, electrochemical sensors are preferred to optical and luminescent sensors because electrochemical sensors are easier to miniaturize, are less costly also owing to the reagents that they use, are faster and more sensitive, can be used also in turbid media, and in many cases provide a better performance.

In a glucometer, the (electrochemical) sensor has an exposed portion treated with specific enzymes so as to interact with the glucose contained in the blood of the patient (e.g., by the glucose-oxidase reaction) and thereon supply an electrical signal that indicates the glucose concentration in blood. The cannula has dimensions such as to enable subcutaneous introduction of the sensor to enable contact with the blood flow.

Normally, the cannula is housed within a casing, which contains also a card carrying the processing unit and the contacts for the sensor. At the moment of use, the cannula is extracted and stretched out for subcutaneous insertion.

For this reason, sensors of glucometers (and many biosensors in general) must have reduced dimensions to enable housing within the cannula, and be strong and at the same time flexible.

Thus, electrochemical sensors have been provided on flexible and compact polymeric substrates, which are generally satisfactory from the mechanical standpoint.

However, the instruments that use biosensors of this type are affected by a certain imprecision in measurement because of the marked dependence of the glucose-oxidase reaction upon temperature. A variation in temperature of a few degrees on the working electrode of the sensor may give rise to variations in the response of the sensor of the order of tens of nanoamps, even for relatively low glucose concentrations. As the concentration increases, the variability of the response is even greater.

Some devices use a temperature sensor within the casing that contains the card with the processing unit. In general, however, the values detected in this way are basically determined by the environmental conditions and represent an approximation of the temperature of the blood flow or of the specimen taken that is excessively crude to allow effective compensation of the dependence of the response upon the temperature.

Other devices use sensors for detecting the temperature on the skin surface. This solution is likewise unsatisfactory because the correlation between the skin temperature and the blood temperature is rather weak. Estimates of this type are hence not sufficient to significantly improve the precision of measurement of known glucometers.

The aim of the present invention is to provide a sensor assembly and a process for manufacturing a sensor assembly that allow one or more of the above limitations to be overcome.

### SUMMARY OF THE INVENTION

According to the present disclosure, a sensor assembly and a process for manufacturing a sensor assembly are provided, as defined, respectively, in claims 1 and 15.

### BRIEF DESCRIPTION OF THE DRAWINGS

For a better understanding of the invention, some embodiments thereof will now be described, purely by way of non-limiting example and with reference to the attached drawings, wherein:

FIG. 1 is a simplified block diagram of a biomedical device incorporating a sensor assembly according to one embodiment of the present disclosure;

FIG. 2 is a top plan view of the sensor assembly of FIG. 1;

FIG. 3 is a cross section through the sensor assembly of FIG. 2, taken along the line III-III of FIG. 2;

FIG. 4 is a top plan view of the sensor assembly of FIG. 2, sectioned along the line IV-IV of FIG. 3;

FIG. 5 is a top plan view of the sensor assembly of FIG. 2, sectioned along the line V-V of FIG. 3;

FIG. 6 is a cross section through a dielectric body in an initial step of a process for manufacturing a sensor assembly according to one embodiment of the present disclosure;

FIG. 7 is a top plan view of the body of FIG. 6;

FIG. 8 shows the view of FIG. 7 in a subsequent operating step;

FIGS. 9 and 10 show the view of FIG. 6 in respective subsequent operating steps;

FIG. 11 is a cross section through a sensor assembly according to a further embodiment of the present disclosure;

FIG. 12 is a cross section through a sensor assembly according to a further embodiment of the present disclosure;

FIG. 13 is a cross section through a sensor assembly according to a further embodiment of the present disclosure;

FIG. 14 is a top plan view of the sensor assembly of FIG. 13, sectioned along the line XIV-XIV of FIG. 13;

FIG. 15 is a top plan view of the sensor assembly of FIG. 13, sectioned along the line XV-XV of FIG. 13; and

FIG. 16 is a top plan view of the sensor assembly of FIG. 13, sectioned along the line XVI-XVI of FIG. 13.

### DESCRIPTION OF EMBODIMENTS OF THE INVENTION

The present invention finds advantageous application in the sector of biosensors, in particular for providing measurements in vivo. In particular, the invention has application in manufacturing glucometers, which are used to exemplify the invention in the following discussion. This must not, however, be considered limiting, in so far as the invention can be exploited for obtaining other types of biosensors, as well as systems of sensors that integrate actuators designed for applications that depart from the biological and biomedical sector.

Examples of biosensors that may exploit the invention include sensors for biomarkers, CO₂ sensors, humidity sensors, dew-point sensors, temperature and pressure sensors for neonatal catheters, temperature and pH sensors for biological specimens, sensors for “smart” plasters or bandages that include also therapeutic systems such as heaters and piezoelectric actuators. Sensors that may be used in other sectors, include, for example, sensors for cold-chain management in transport and logistics of foodstuff products, and sensors for control of integrity of pharmaceutical products, and the like.

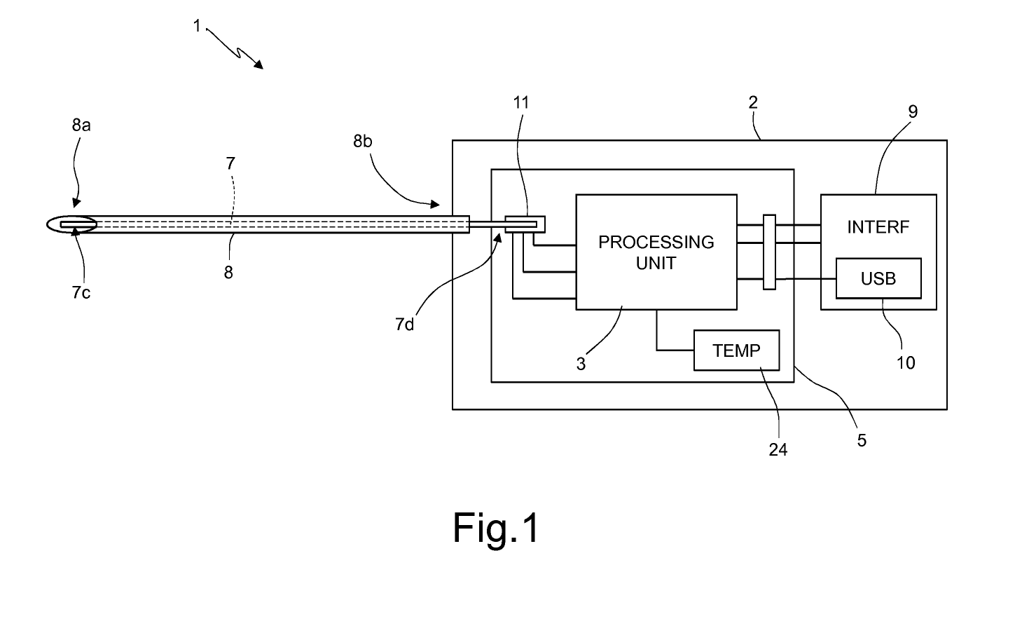

In FIG. 1, a glucometer according to one embodiment of the present disclosure is designated as a whole by the reference number 1. The glucometer 1 comprises a casing 2, which houses a processing unit 3 mounted on a card 5, and a sensor assembly 7, in part housed in a cannula 8, designed for hypodermic introduction. Moreover, an interface 9, which enables reception of commands from a user for execution of measurements and display of the results, is connected to the processing unit 3. In one embodiment, the interface 9 is provided with a communication port 10 (for example, a USB port) for connection with an external processing system, here not shown.

The cannula 8 is preferably made of polymeric material, is flexible, and is sufficiently thin as to be used as needle. Prior to use, the cannula 8 is folded or contained within the casing 2 to reduce the overall dimensions of the glucometer 1 and is extracted and stretched out for being inserted through the skin of a patient. In FIG. 1, the cannula 8 is represented in the stretched-out configuration.

The sensor assembly 7 basically occupies the entire length of the cannula 8 and is accessible by fluids through a distal end 8a to enable the measurements. The sensor moreover projects from an opposite proximal end 8b of the cannula so as to be connected to the processing unit 3 through a connector 11 on the card 5.

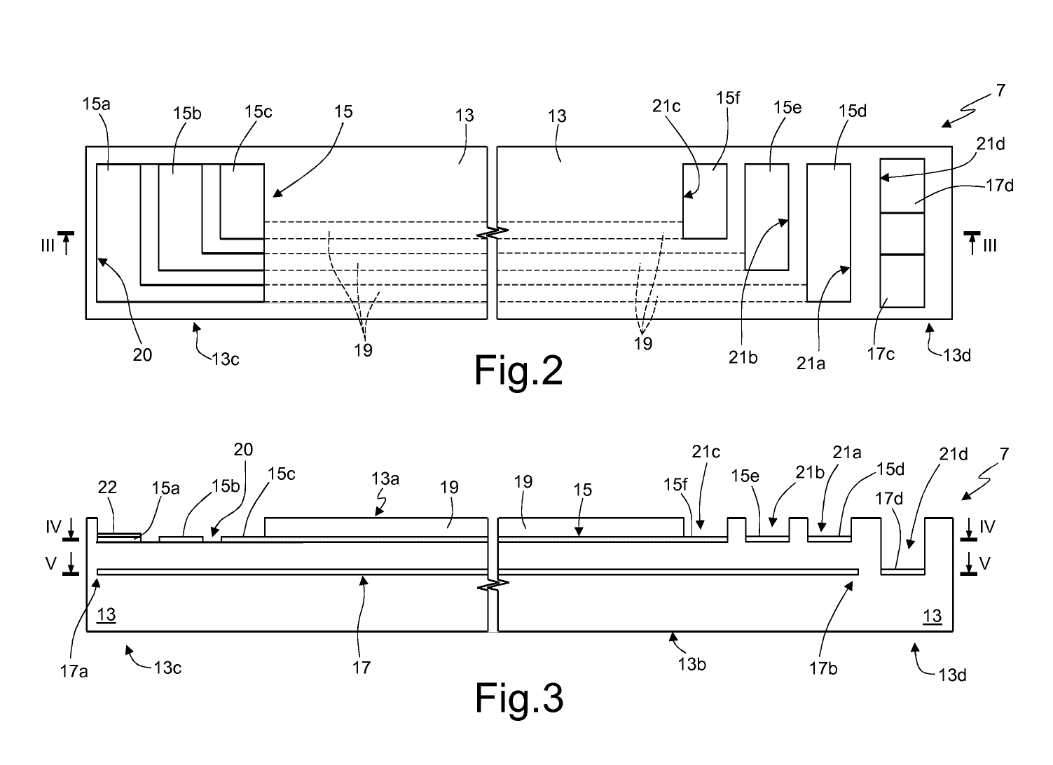

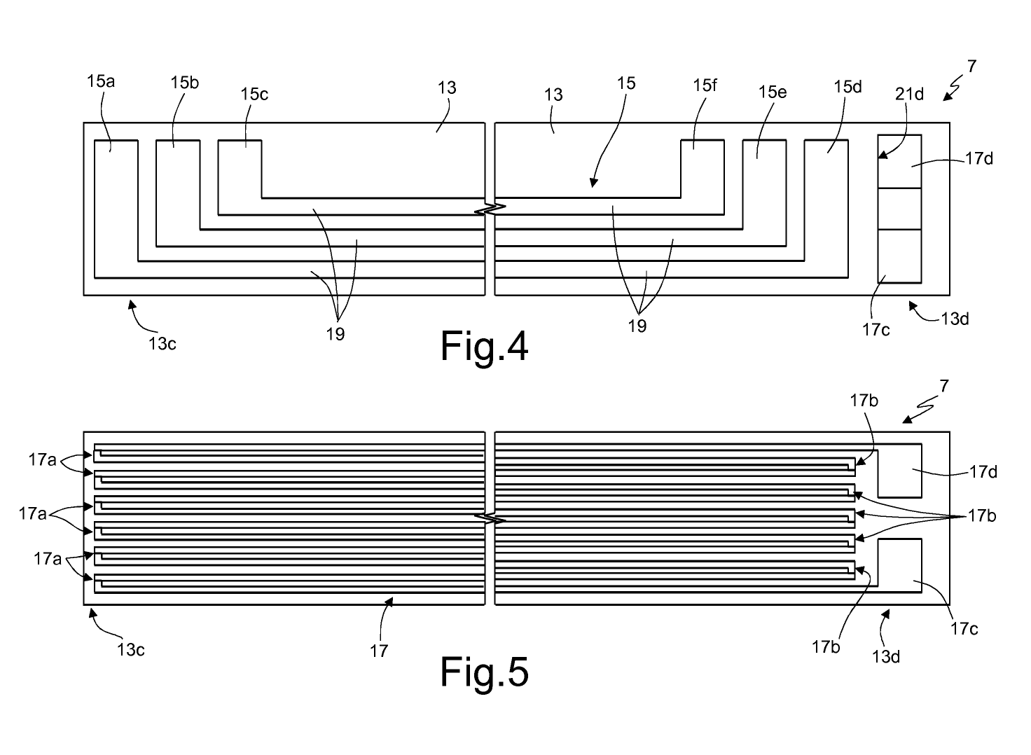

FIGS. 2-5 show in greater detail the sensor assembly 7, which comprises a monolithic body 13, a biosensor 15, and a temperature sensor 17.

The body 13 of the sensor assembly 7 is an elongated planar plate of a substantially rectangular shape and has a first main face 13a and a second main face 13b opposite to one another (FIG. 3). The body 13 is made of a dielectric polymeric material and is flexible. In one embodiment, in particular, the body 13 is made of polyimide. Alternatively, it is possible to use also other polymers such as, for example, polypropylene (PP), polyethylene (PE), polystyrene (PS), polymethylmethacrylate (PMMA), polyvinyl chloride (PVC), and also conjugated polymers such as poly(3-hexylthiophene) (P3HT) and MEH-PPV, to name a few.

The biosensor 15 and the temperature sensor 17 are planar and located in respective distinct planes so as to overlap and are both incorporated in the body 13. In particular, the biosensor 15 is arranged between the first main face 13a of the body 13 and the temperature sensor 17. The temperature sensor 17 is arranged between the biosensor 15 and the second main face 13b of the body 13.

In the embodiment described, the biosensor 15 is of an amperometric electrochemical type and is coated by a portion of the body 13 that functions as passivation layer.

The biosensor 15 comprises a working electrode 15a, a reference electrode 15b, and a counterelectrode 15c, which are electrically insulated from one another and connected to respective contact pads 15d, 15e, 15f through connection lines 19 incorporated in the body 13. The working electrode 15a, the reference electrode 15b, and the counterelectrode 15c are arranged at one end 13c of the body, which is in turn set in the proximity of the distal end 8a of the cannula 8 (see FIG. 1).

The working electrode 15a, the reference electrode 15b, and the counterelectrode 15c are accessible from outside through a measurement window 20 in the portion of the body 13 that coats the biosensor 15 and functions as passivation layer (FIGS. 2 and 3).

The contact pads 15d, 15e, 15f are located at one end 13d of the body 13 that is opposite to the end 13c and projects from the proximal end 8b of the cannula 8 for coupling to the connector 11 (FIG. 1). The coupling is provided through contact windows 21a, 21b, 21c in the body 13 (FIGS. 2 and 3).

The working electrode 15a, the reference electrode 15b, and the counterelectrode 15c are made, for example, of gold, platinum, or silver. In addition, the working electrode 15a is functionalized by a layer of e.g., silver paste 22, immobilized on which are enzymes. In the specific case of detecting blood glucose, the immobilized enzymes catalyze the process of oxidation of blood glucose, but other enzymes can be used for different applications and such enzymes are responsible for the specificity of the sensor.

The temperature sensor 17 is incorporated in the body 13 at a distance from the first main face 13a such as to guarantee electrical insulation and, at the same time, a good thermal coupling with respect to the biosensor 15, at least in a region corresponding to the measurement window 20.

In the embodiment described herein, the temperature sensor 17 is of a differential type and co-operates with an auxiliary temperature sensor 24 (FIG. 1), for example arranged on the card 5 in the casing 2. In particular, the temperature sensor 17 is a thermopile that includes a plurality of thermocouples connected in series and has hot junctions 17a (sensing elements) set at the end 13c of the body 13 and thermally coupled to the electrodes 15a, 15b, 15c of the biosensor 15 (in particular, to the working electrode 15a). Cold junctions 17b (reference elements) and contact pads 17c, 17d of the temperature sensor 17 are set at the end 13d of the body 13. The contact pads 17c, 17d are accessible for connection to the connector 11 of the card 5 through a contact window 21d in the body 13.

In alternative embodiments (not shown), instead of the thermopile, a different temperature sensor can be used, such as for example a thermistor or a thermoresistance sensor. In any case, the sensitive element is arranged in the proximity of the electrodes 15a, 15b, 15c of the biosensor 15, in particular in the proximity of the working electrode 15a so as to be thermally coupled thereto.

In use, the glucometer 1 exploits the information supplied by the temperature sensor 17 of the biosensor 15 to compensate for the dependence upon the temperature of the response of the electrodes 15a, 15b, 15c. In the embodiment described, in particular, the temperature sensor 17 supplies a first temperature signal ST1 indicative of the difference in temperature between the hot junctions 17a (which are at the temperature of the electrodes 15a, 15b, 15c of the biosensor 15) and the cold junctions 17b (which are at ambient temperature). The processing unit 3 adds the first temperature signal ST1 to a second temperature signal ST2 supplied by the auxiliary temperature sensor 24 and indicative of the ambient temperature in the casing 2, where the cold junctions 17b of the temperature sensor 17 are located.

The sensor assembly 7 described basically enables elimination of the errors of measurement due to an imprecise compensation of the dependence upon the temperature of the response of the biosensor 15, without increasing the dimensions of the device significantly. The temperature sensor 17 incorporated in the body 13 of the sensor assembly 7 is in fact stacked on a different level with respect to the biosensor 15 and requires a thickness of just a few microns, whilst the overall area occupied basically coincides with the area of the biosensor 15. The degree of miniaturization and the mechanical properties (in particular the flexibility) of the sensor assembly 7 are hence not altered by the presence of the temperature sensor 17.

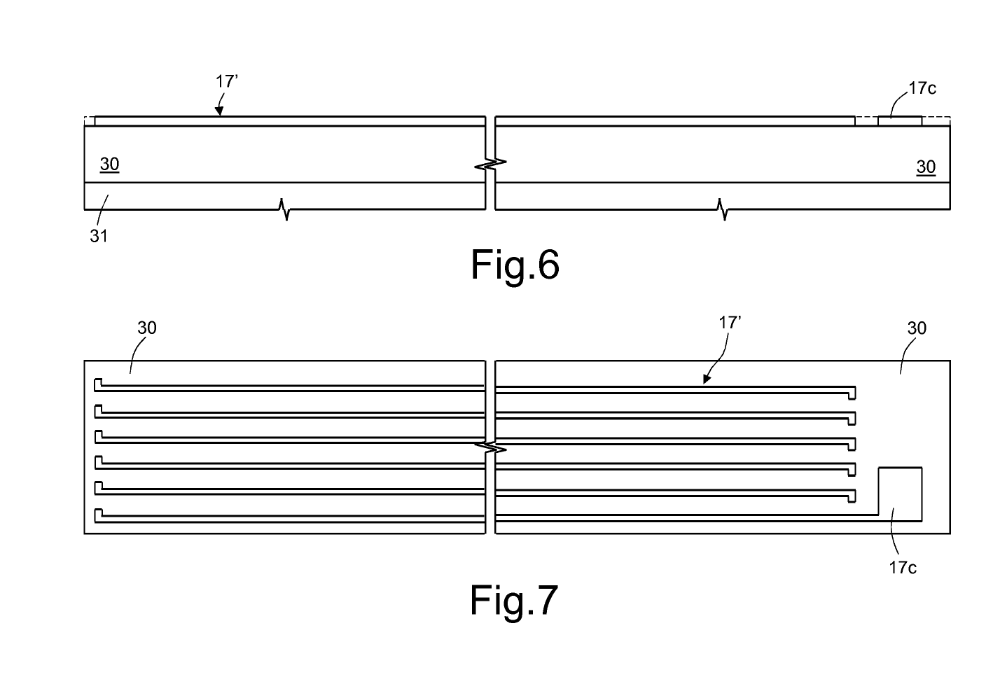

FIGS. 6-10 illustrate a process for manufacturing the sensor assembly 17.

Initially, a substrate 30 of dielectric polymeric material is formed on a rigid support 31, for example a wafer of a substantially circular shape. The substrate 30 has a thickness such as to be flexible, for example 20 μm. The polymeric material, here polyimide, is deposited for example by a physico-chemical spin-coating process, which enables precise control of the thickness of the structures obtained. Alternatively, techniques of continuous roller deposition can be used, such as, for example, slot coating and die coating. In these cases, the support 31 is in the form of rectangular panel.

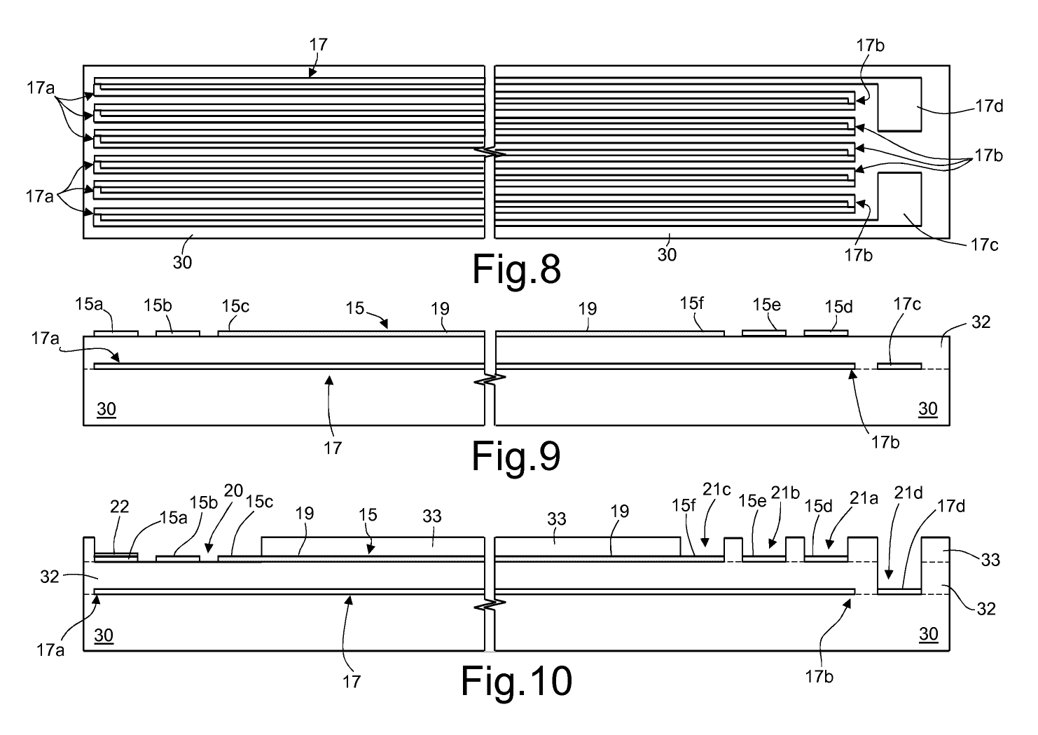

Next, a first metallization layer (first junction material) is deposited and patterned by a first photolithographic process to obtain a first portion 17′ of the temperature sensor 17 and the contact pad 17c, as shown in FIG. 7. The temperature sensor 17 is completed by deposition of a second metallization layer (second junction material) and a second photolithographic process (FIG. 8). The first junction material and the second junction material are selected so as to produce a difference of potential by the Seebeck effect.

The substrate 30 and the temperature sensor 17 are then coated with an intermediate layer 32, for example, having a thickness of 3 μm (FIG. 9). The intermediate layer 32 is deposited directly on the substrate 30 with the same technique (spin coating, in this case) and is made of the same material (polyimide).

A third metallization layer is then deposited and patterned by a third photolithographic process to obtain the biosensor 15. In this step, a mask (not shown) is used, which enables alignment of the working electrode 15a of the biosensor 15 to the hot junctions 17a of the temperature sensor 17.

A passivation layer 33 is then deposited directly on the intermediate layer 32 and coats the biosensor 15 (FIG. 10). The passivation layer 33 is made of the same material of which also the substrate 30 and the intermediate layer 32 are made and, moreover, the same technique is used, here the spin-coating technique. The substrate 30, the intermediate layer 32, and the passivation layer 33 thus form the body 13, which is homogeneous and continuous and incorporates the temperature sensor 17.

Finally, the measurement window 20 and the contact windows 21a, 21b, 21c, 21d are opened, e.g., by etching. The contact assembly illustrated in FIGS. 2-5 is thus obtained.

The process described presents the advantage of bestowing homogeneity and continuity on the body 13, which can thus present a high degree of flexibility in a compact structure and has a low risk of detachment of layers formed in successive steps. Moreover, the body 13 does not require intermediate adhesion layers, which in addition to worsen the mechanical properties, present serious problems from the standpoint of biocompatibility.

In some embodiments, the substrate, the intermediate layer, and the passivation layer may be made of different materials, which are preferably deposited directly on top of one another using the same technique, in particular the spin-coating technique, which does not envisage the use of auxiliary adhesion layers.

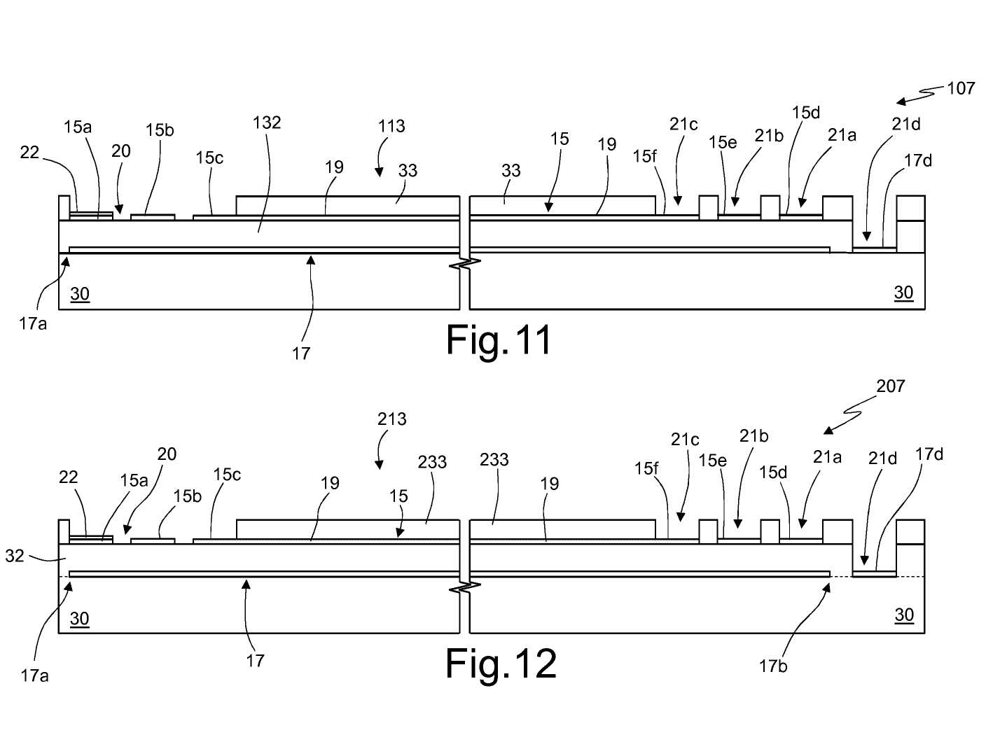

For example, in the embodiment of FIG. 11, where parts that are the same as the ones already described are designated by the same reference numbers, in a sensor assembly 107 a body 113 comprises an intermediate layer 132 made of alumina between the substrate 30 and the passivation layer 33, which are made of polyimide. The use of alumina presents the advantage of enabling an excellent thermal coupling between the biosensor 15 and the temperature sensor 17, to the advantage of the measuring precision.

In the embodiment of FIG. 12, in a sensor assembly 207 a body 213 comprises a passivation layer 233 made of a polymer different from the one forming the substrate 30 and the intermediate layer 32.

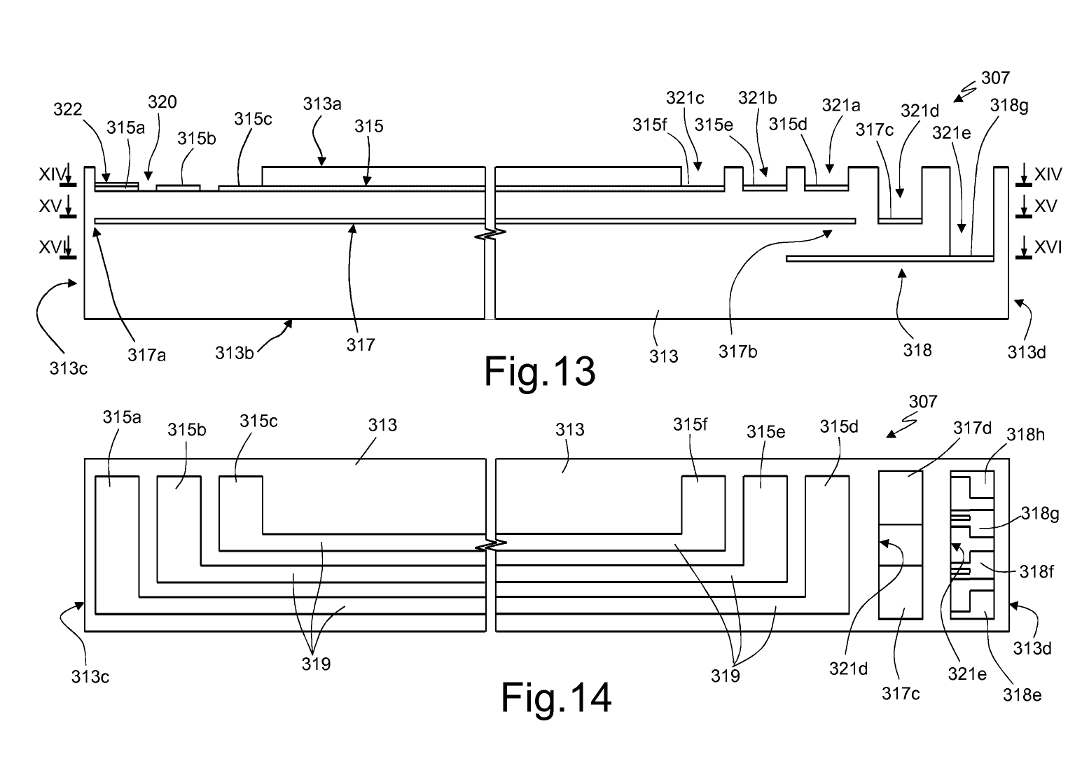

FIGS. 13-16 show a sensor assembly 307 according to a further embodiment of the invention. The sensor assembly comprises a body 313, a biosensor 315, a primary temperature sensor 317, and an auxiliary temperature sensor 318.

The body 313 of the sensor assembly 307 is an elongated planar plate of a substantially rectangular shape and has a first main face 313a and a second main face 313b opposite to one another. The body 313 is made of a dielectric polymeric material, for example polyimide, and is flexible.

The biosensor 315, the primary temperature sensor 317, and the auxiliary temperature sensor 318 are planar and located in respective distinct planes so as to overlap at least partially, and are all incorporated in the body 313. In particular, the biosensor 315 is arranged between the first main face 313a of the body 313 and the temperature sensor 317, the temperature sensor 317 is arranged between the biosensor 315 and the auxiliary temperature sensor 318, and the auxiliary temperature sensor 318 is arranged between the temperature sensor 317 and the second main face 313b of the body 313.

The biosensor 315 (FIGS. 13 and 14) is of an amperometric electrochemical type, substantially of the type already described with reference to FIGS. 2-5. In particular, the biosensor 315 comprises a working electrode 315a, a reference electrode 315b, and a counterelectrode 315c, which are electrically insulated from one another and connected to respective contact pads 315d, 315e, 315f through connection lines 319 incorporated in the body 313.

The working electrode 315a is functionalized by a layer of silver paste 322, immobilized on which are enzymes for interaction with the blood glucose in the diabetes application herein described, but can be another enzyme for a different application. The working electrode 315a, the reference electrode 315b, and the counterelectrode 315c are set at one end 313c of the body 313 and are accessible from outside through a measurement window 320 in the portion of the body 313 that coats the biosensor 315 and functions as passivation layer.

Contact windows 321a, 321b, 321c in the body 313 enable electrical coupling of the contact pads 315d, 315e, 315f, which are set at one end 313d of the body 313 opposite to the end 313c (FIG. 13).

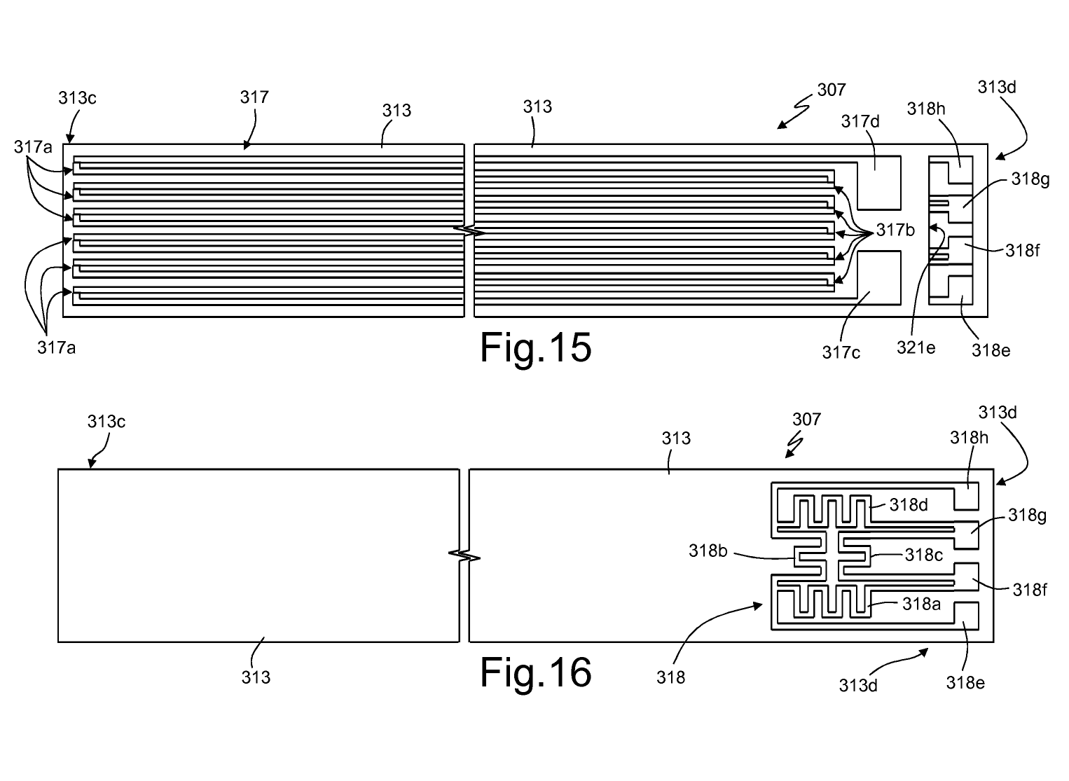

The primary temperature sensor 317 (FIG. 15) is a thermopile, substantially as already described with reference to FIGS. 2-5. The temperature sensor 317 includes a plurality of thermocouples connected in series and has hot junctions 317a (sensing elements) arranged at the end 313c of the body 313 and thermally coupled to the electrodes 315a, 315b, 315c of the biosensor 315 (in particular to the working electrode 315a) (seen in FIG. 13).

Cold junctions 317b (reference elements) and contact pads 317c, 317d of the temperature sensor 317 are arranged at the end 313d of the body 313. The contact pads 317c, 317d are accessible for electrical connection through a contact window 321d, provided in the body 313 (FIG. 13).

The auxiliary temperature sensor 318 (FIG. 16) supplies an absolute temperature measurement and is thermally coupled to the cold junctions 317b of the primary temperature sensor 317 of FIG. 15. In the embodiment described here, the auxiliary temperature sensor 318 is of the thermistor-bridge type (in FIG. 16 four thermistors 318a, 318b, 318c, 318d are shown). Contact pads 318e, 318f, 318g, 318h are accessible for electrical coupling through a contact window 321 e in the body 313 (FIG. 13).

The auxiliary temperature sensor 318 enables an accurate measurement of the temperature at the cold junctions 317b of the primary temperature sensor 317 and, consequently, of the temperature at the electrodes 315a, 315b, 315c of the biosensor 315.

Modifications and variations may be made to the sensor assembly and to the process described, without thereby departing from the scope of the present invention, as defined in the annexed claims.

What is claimed is:

1. A sensor assembly comprising:
a) an electrically insulating and flexible body, in the form of a plate having a first face and a second face; and
b) a first sensor and a second sensor, incorporated in the body:
c) wherein the first sensor is arranged between the first face and the second sensor and the second sensor is arranged between the first sensor and the second face.

2. The sensor assembly according to claim 1, wherein the first sensor and the second sensor overlap at least partially.

3. The sensor assembly according to claim 1, wherein the first sensor and the second sensor are planar sensors.

4. The sensor assembly according claim 1, wherein the second sensor is operatively coupled to the first sensor.

5. The sensor assembly according to claim 4, wherein the first sensor comprises a first sensing element and the second sensor comprises a second sensing element operatively coupled to the first sensing element.

6. The sensor assembly according to claim 1, wherein the first sensor is a biosensor and comprises sensing electrodes arranged at a first end of the body; and the second sensor is a temperature sensor thermally coupled to the sensing electrodes.

7. The sensor assembly according to claim 6, wherein the first sensor comprises contact pads arranged at a second end of the body, opposite to the first end, and wherein the contact pads are connected to respective sensing electrodes through connection lines incorporated in the body.

8. The sensor assembly according to claim 7, wherein the first sensor is an electrochemical sensor and the second sensor comprises a thermopile having hot junctions thermally coupled to the sensing electrodes and cold junctions arranged at the second end of the body.

9. The sensor assembly according to claim 1, comprising a third sensor incorporated in the body, operatively coupled to at least one of the first sensor and the second sensor and arranged between second sensor and the second face of the body.

10. The sensor assembly according to claim 9, wherein the third sensor is thermally coupled to the cold junctions of the second sensor.

11. The sensor assembly according to claim 1, wherein the body is homogeneous.

12. The sensor assembly according to claim 11, wherein the body is made of a single material selected from the group consisting of: polyimmide, polypropylene, polyethylene, polystyrene, polymethylmetacrylate, polyvinyl chloride, poly (3-hexylthiophene), MEH-PPV.

13. A glucometer, comprising a sensor assembly according to claim 1.

14. The glucometer according to claim 13, comprising a casing and a cannula extractable from the casing; wherein the sensor assembly is accommodated in the cannula and is accessible for measures through one end of the cannula.

15. A process for manufacturing a sensor assembly having a body, a first sensor and a second sensor incorporated in the body, the method comprising:
a) forming a first layer that is electrically insulating and flexible;
b) forming the second sensor on the first layer;
c) forming a second layer on the first layer, so as to incorporate the second sensor;
d) forming the first sensor on the second layer;
e) forming a third layer on the second layer, so as to incorporate the first sensor.

16. The process according to claim 15, wherein the first layer, the second layer and the third layer are made of a single material selected from the group consisting of: polyimmide, polypropylene, polyethylene, polystyrene, polymethylmetacrylate, polyvinyl chloride, poly (3-hexylthiophene), and MEH-PPV.

17. The process according to claim 15, wherein the first layer, the second layer and the third layer are made using one and the same deposition technique.

18. The process according to claim 15, wherein forming the first sensor and forming the second sensor comprise depositing and defining respective metallization layers through photolithographic processes.

19. A blood sensor comprising:
a. a hypodermic cannula containing a flexible sensor assembly therein,
b. said flexible sensor assembly comprising a polymeric body with an embedded planar biosensor and an embedded planar temperature sensor in said body,
c. wherein each of said biosensor and said temperature sensor are located in respective distinct planes in said body so as to at least partially overlap,
d. wherein said body is homogeneous and does not comprises layers bonded together with an adhesive, and
e. wherein said biosensor has an enzyme immobilized thereon.

20. The blood sensor of claim 19, wherein said enzyme is glucose oxidase.
